(19) United States
(12) Patent Application Publication
(10) Pub. No.: US 2006/0078546 A1
(43) Pub. Date: Apr. 13, 2006

(54) Use of lactic acid bacteria to reduce sick time

(76) Inventors: Anders Zachrisson, Landskrona (SE); Bo Mollstam, Lerum (SE)

Correspondence Address: Laynn E. Barber, Post Office Box 16528, Fort Worth, TX, 76162, US

(21) Appl. No.: 10/960,126

(22) Filed: Oct. 7, 2004

(51) Int. Cl. A61K 35/74

(52) U.S. Cl. 424/93.45

(57) ABSTRACT

A method of decreasing sick time, and thus sick-leave, in which selected strains of lactic acid bacteria are administered.

### BACKGROUND OF THE INVENTION

1. Field of the Invention

This invention relates to the use of selected lactic acid bacteria to increase the nutritional health status of people to support their well-being and thereby directly decrease the time such people are unable to work due to sickness.

2. Description of the Related Art

The cost of sick-leave, treatment, rehabilitation and early retirement is sky-rocketing in most countries. Most attention is to a large extent focused on long-term disease and how to overcome the economical burden this puts on the society as a whole. However, not generally known is that sick-pay for short-term (<3 weeks) absence from the work, for example, in Sweden, costs more than 20 billion SEK per year. Significant costs for short-term absence from work is also found in other countries. Apart from the cost itself, short-term absence is disruptive to production as it is almost impossible to plan for these absences.

The general well-being in work-places is receiving increased attention. Not only is the well-being and healthiness important to the individual himself, but also important to fellow co-workers, family members and last but not least to the success of the company. During the last few decades increased focus have been on how to increase well-being by offering company-sponsored health care, memberships in fitness centres and similar programs aiming to prevent and cure disease and to increase physical activity and awareness on health issues.

Comparatively little attention have been on the diet and how a daily healthy diet impacts on general health with the exception of anti-obesity initiatives. Studies of people with irregular work and eating habits show that such behaviour correlates with increased risk for various diseases. Diet initiatives for the non-obese and general work-force, occasionally under various kinds of stress, has not been a focus so far. Disrupted sleep and eating patterns interfere with nutritional utilization. Disruptions can result in sleep and stress-related disorders and may also affect the cardiovascular system.

Gastrointestinal problems, particularly ulcers, are more common among shift workers. Factors that may contribute to these problems include that shift workers may eat less at night, while others may over-eat, consuming night time snacks in addition to regular day meals and that shift workers often eat at night when digestion and other body functions are slowed down (Waterhouse et al, “Shiftwork, health and safety”, HSE Contract Research Report No 31/1992). The same apply to other people with irregular work and eating habits. For example an accumulated sleep deficit may leave a person more vulnerable to stress-related disorders.

The healthy gastrointestinal tract (gut) performs a multitude of functions. It digests foods; absorbs small food particles that are converted into energy; transports vitamins and minerals across the intestinal lining and into the bloodstream; contributes to the chemical detoxification system of the body. It has previously been described (Wilson L M, et al, Microcirculation. 1999 September;6(3): 189-98) that stress also can alter the physiological characteristics of gastrointestinal structures, such as mucosal villi and underlying crypts, in for example stress-related damages of such cells. Mammalian cell divide at various rates. Generally for a group of cells that rarely complete the cell cycle, we expect a high proportion of cells to be in the resting stage of the cell cycle. However, in a rapidly dividing cell population, such as in GI-tract villi, structures we expect a high proportion of cells to be in the stage of mitosis. One way to quantify cell division is by using the mitotic index. The so called “mitotic index” can also be used to quantify differences in cell division when an environmental parameter is changed. By quantifying properties of cell division, we can compare differences in cell growth among neighbouring cells and in response to environmental variables.

The mitogenic effect of selected lactic acid bacteria in the present invention have a beneficial effect(s) in everyday situations such as repairing damage villi cells for improved utilization of important nutrients such as vitamins, minerals and other essential food components to counteract the effects of stress on people. Stress is here to stay, but there are effective methods for minimizing the damage it can cause. In addition to stress awareness and lifestyle changes, certain dietary supplements, including vitamins, minerals and herbal products, can support the body's ability to handle stress. Researchers are finding that many supplements may help manage and treat short-term stress and stress-related illness. The use of selected lactic acid bacteria to aid in the production of butyrate to better utilize such nutrients and supplements is the essence of the present invention.

It has previously been described that short chain fatty acids (SFA), especially butyric acid, can aid in the regeneration of villi structures in the gut, Others have also reported that some, but not all, lactic acid bacteria can be part of the butyric acid production in the intestine as one mechanism to combat pathogens (M. Banasaz et al, Increased Enterocyte Production in Gnotobiotic Rats Mono-Associated with Lactobacillus rhamnosus GG, Applied and Environmental Microbiology, June 2002, p. 3031-3034, Vol. 68, No. 6).

Butyrate, or butyric acid, has previously been used for example at the University of Illinois to show that patients who have had parts of their small intestine surgically removed can be helped by addition of it to an intravenous nutrition solution, this may not only cause the intestine to grow but makes it more functional as well. (Tappenden et al, 2004 Harry M. Vars Award July/August issue of the Journal of Parenteral and Enteral Nutrition).

During the last decade there has been an increasing interest in so called probiotics, bacterial cultures with claimed health benefits. Today there is a large number of different probiotic products available either as ingredients in “functional foods” or as dietary supplements. For example, the probiotic Lactobacillus reuteri is a major component of the lactobacilli population, which naturally inhabit the gastrointestinal tract of humans and animals. The organism has been extensively studied as a probiotic over the last ten years and found to possess a number of interesting properties, including inhibition of pathogens and modulation of the immune system.

It has now been surprisingly found that specific lactic acid bacteria, selected for their ability to aid in intestinal butyric acid production and administered to generally healthy people, occasionally under stress, will directly influence the nutritional health status of such people and reduce their incidences of sickness.

It is therefore an object of the invention to provide such a method for decreasing illnesses, and thereby increase work-place healthiness.

Other objects and advantages will be more fully apparent from the following disclosure and appended claims.

### SUMMARY OF THE INVENTION

The invention herein is a method to increase the nutritional health status of people to support their wellbeing and thereby directly resulting in decreased sick time, and thus sick-leave, in which specifically selected strains of lactic acid bacteria are administered.

Other objects and features of the inventions will be more fully apparent from the following disclosure and appended claims.

### DETAILED DESCRIPTION OF THE INVENTION AND PREFERRED EMBODIMENTS THEREOF

The invention herein is a method of decreasing sick time, and thus sick-leave, in which cells of one or more selected strains of lactic acid bacteria are administered, preferably at least 10⁵ to 10⁶ cells per person per day for a period of at least 10 days.

The strains of lactic acid bacteria to be used in the invention are selected by growing them in vitro, then administer the cultures to test subjects and analyzing for butyrate production in fecal material using HPLC, GC or other methods. Alternatively are such lactic acid bacteria strains used already reported by others to well stimulate the production of butyric acid in humans.

The features of the present invention will be more clearly understood by reference to the following examples, which is not to be construed as limiting the invention.

### EXAMPLE 1

The aim of this study was to determine if a daily health-snack with an added lactic acid bacteria helping in the intestinal production of butyrate, can further improve work-place healthiness assessed as illnesses occurring in volunteers after the start of the study.

a. Selection of Strain

The lactic acid bacteria strain to be used is selected for it's ability to produce or aid in the production of butyric acid in humans.

Bacterial Strains

6 strains of lactic acid bacteria are studied, They are taken from ATCC (Baltimore, Md., US) and identification confirmed using biochemical methods (API system, Biomerieux, Craponne, France).

The strains are cultured in MRS media (Oxoid, Ogdensburg, N.Y., USA) for 2 days, then harvested, and washed and given to test subjects.

Acid Analysis

Faecal samples are collected from the test subjects at day 0 before start of the study and for 4 days after administering the test strains. Fresh faecal samples (5.0 g) are acidified and diluted with 5.0 mL of 25% m-phosphoric acid and 15.0 mL of distilled water. After 30 min, samples are centrifuged at 25,000×g for 20 min. The supernatant fluid is aspirated into microfuge tubes and frozen at −20° C. Amounts of butyrate is determined using a Hewlett-Packard 5890A Series II gas chromatograph and glass column (180 cm×4 mm i.d.) packed with 10% SP-1200/1% H3PO4 on 80/100 mesh Chromsorb W AW (Supelco Inc., Bellefonte, Pa.). Nitrogen is used as the carrier gas with a flow rate of 75 mL/min. Oven, detector, and injector temperatures are 125, 175, and 180° C., respectively.

Results

The strains resulting in the highest difference in butyric acid between day 0 and the test days are selected. In this case, a Lactobacillus reuteri strain, ATCC 55730, is selected.

b. Confirmation of Effect on Sick-Leave

In this study healthy volunteers were recruited among employees at Tetra Pak in Lund and Sunne, Sweden. The criteria for participation were that they should be between 18-65 years of age, willing to comply with the protocol and that signed informed consent was obtained. The study was subject to approval by the ethical committee at Lund University Hospital.

The overall study period was set to 84 days. The volunteers were randomised in a double-blind fashion to receive a daily drink-snack with a straw containing the selected L. reuteri strain by the method above in 1.a or an identical straw with placebo. The L. reuteri straw is manufactured according to the specification of U.S. Pat. No. 6,283,294, the disclosure of which is incorporated by reference herein. The placebo straw is manufactured in exactly the same manner, except that there are no lactobacilli added to the carrier oil in the straw.

During the study period the volunteers were asked to report illness symptoms related to the respiratory tract and/or the gastrointestinal tract, as well as if these symptoms resulted in sick-leave or not, and if so, the duration of the sick-leave.

Results

262 volunteers entered the study after ethical committee approval had been obtained. 38 were excluded from further analysis as they were reported to be ill at study start. Another 5 were excluded since their compliance was assessed as being too low, they had used the study product for less than 28 days. Of the remaining 219, 108 were randomised to receive a snack supplemented with a placebo-containing straw and 111 to receive a snack fortified with a L. reuteri-containing straw. At the time of analysis it was decided to exclude 3 volunteers from the placebo group and 2 from the reuteri group as these volunteers had consumed the study product for less than 28 of the stipulated 84 days. These post-study exclusions did not impact on study outcome measures.

Data were analysed at 2 different levels:
Level 1: requirement that the subjects should have used the study products during at least 28 days. (placebo: n=108, reuteri: n=111)
Level 2: additional requirement that the subjects should have used the study products during at least 80 days and should not have “missed” study treatment for more than 7 days (placebo: n=83, reuteri: n=91)

The results for the main outcomes in the study are depicted in table 1.

TABLE 1
Main outcome assessments

| | Level 1 | | Level 2 | |
|---|---|---|---|---|
| | Placebo | L. reuteri | Placebo | L. reuteri |
| No. of subjects | 108 | 111 | 83 | 91 |
| No. of subjects | 25(23%) | 14(13%) | 21(25%) | 10(11%) |
| reporting sick-days | | | | |
| Significance | P < 0.05 | | P < 0.05 | |
| Frequency* of | 0.64% | 0.59% | 0.9% | 0.3% |
| sick-days | | | | |

| | | |
|---|---|---|
| Significance | p < 0.05 | p < 0.01 |

*number of sick days divided by total number of days in the study

A total of 355 symptom episodes were reported. The most common were common cold 28%, running nose 24%, sore throat 22%, fever 14%, cough 12% and fatigue 12%.

Discussion

The present study was performed at two different Tetra Pak locations, Lund and Sunne in Sweden during the late winter and spring season when short time absence is relatively common, mostly due to respiratory tract ailments such as the common cold but also a diversity of GI problems.

The results obtained demonstrates that a daily health snack fortified with L. reuteri according to the invention herein reduces the number of individuals reporting sick by half. The effect of the L. reuteri fortified health snack furthermore reduces the risk of becoming ill by ⅔ and the frequency of sick-days is reduced from 0.9% of ordinary working days down to 0.3%.

The difference between Level 1 and Level 2 was that the compliance in the latter group was higher as it was required that they had consumed the study product at least 80 days vs 28 days in the Level 1 group. The observed differences in study outcome between these supports the notion that a long-term intake is better than short-time intake. There was also improved effect in the sub-group working shifts, showing the importance of the invention for people under stress and irregular working and eating habits.

Conclusions from the study are that it is advisable for companies to offer their employees a daily health snack fortified with L. reuteri both for the benefit of the employees as well for the company as costs and production problems due to short-term absence can be reduced.

### EXAMPLE 2

The purpose of this example is to show the benefit of using the present invention in a business method for increasing the effectiveness of a machine (technical equipment) handled by human beings characterised in that the selected L. reuteri non-medical supplement is produced or obtained and distributed to the work force to increase the effectively and frequency of the handling.

The strain to be used is selected following Example 1 above. The same strain is selected, Lactobacillus reuteri ATCC 55730, the strain is used to manufacture a drinking straw following the specification of U.S. Pat. No. 6,283,294, incorporated by reference herein. This straw is then distributed freely by the management of a company to it's employees to be used in a suitable drink on a daily basis for a minimum of 10 days. The number of reported cases of sick-leave is significantly reduced.

While the invention has been described with reference to specific embodiments, it will be appreciated that numerous variations, modifications, and embodiments are possible, and accordingly, all such variations, modifications, and embodiments are to be regarded as being within the spirit and scope of the invention.

1. A method of decreasing a person's sick time, comprising:
a) selecting at least one strain of lactic acid bacteria that aids in the intestinal production of butyric acid; and
b) feeding the person at least 10⁶ cells of the selected at least one strain of lactic acid bacteria per day for at least 10 days

2. The method according to claim 1, wherein the person is fed 10⁸ CFU per day for 10 days.

3. The method of claim 1, wherein the strain is Lactobacillus reuteri ATCC55730.

4. (canceled)

5. A product having efficacy in decreasing a person's sick leave, comprising cells of a lactic acid bacteria strain selected by the strain's ability to aid in the intestinal production of butyric acid.

6. A method for increasing performance and efficiency of persons in a workforce, comprising selecting a lactic acid bacteria strain that aids in the intestinal production of butyric acid and distributing cells of the selected lactic acid bacteria strain to the workforce by a business manager.

7. Use of a selected lactic acid bacteria or an agent comprising a selected lactic acid bacteria for it's ability to aid in the intestinal production of butyric acid for increasing the performance and effectiveness of a technical equipment or machinery under the control of humans, whereby the selected lactic acid bacteria or an agent comprising the selected lactic acid bacteria is distributed to the human workforce to increase the efficiency of the control.
